(19) United States
(12) Patent Application Publication
(10) Pub. No.: US 2007/0122540 A1
(43) Pub. Date: May 31, 2007

(54) Coatings on ophthalmic lenses

(75) Inventors: Joseph C. Salamone, Boca Raton, FL (US); Jeffrey G. Linhardt, Fairport, NY (US); Jay F. Kunzler, Canandaigua, NY (US); Daniel J. Hook, Fairport, NY (US); Daniel M. Ammon JR., Penfield, NY (US)

Correspondence Address: Bausch & Lomb Incorporated, One Bausch & Lomb Place, Rochester, NY, 14604-2701, US

(73) Assignee: Bausch & Lomb Incorporated

(21) Appl. No.: 11/604,635

(22) Filed: Nov. 27, 2006

Related U.S. Application Data
(60) Provisional application No. 60/740,533, filed on Nov. 29, 2005.

(51) Int. Cl. A61L 33/00; A61F 2/02

(52) U.S. Cl. 427/2.24; 424/423

(57) ABSTRACT

This invention is directed toward surface treatment of a device. The surface treatment comprises the attachment of terminal functionalized surfactants to the surface of the substrate by means of reactive functionalities of the terminal functionalized surfactant material reacting with complementary surface reactive functionalities in monomeric units along the polymer substrate. The present invention is also directed to a surface modified medical device, examples of which include contact lenses, intraocular lenses, vascular stents, phakic intraocular lenses, aphakic intraocular lenses, corneal implants, catheters, implants, and the like, comprising a surface made by such a method.

### CROSS REFERENCE

This application claims the benefit of Provisional Patent Application No. 60/740,533 filed Nov. 29, 2005 is incorporated herein by reference.

### FIELD OF INVENTION

The present invention relates generally to reactive surfactants and compositions comprising the surfactants as covalently bound coatings used in the manufacture of medical devices. More specifically, the present invention relates to surface coated ophthalmic lenses formed from one or more functionalized poloxamers or poloxamines having reactive functionality that is complimentary to surface functionality of the ophthalmic lens.

### BACKGROUND OF THE INVENTION

Poloxamer block copolymers are known compounds and are generally available under the trademark PLURONIC. Poloxamers generally have the following structure:
HO(C₂H₄O)ₐ(C₃H₆O)_{b}(C₂H₄O)ₐH

Reverse poloxamers are also known block copolymers and generally have the following structure:
HO(C3H₆O)_{b}(C₂H₄O)ₐ(C₃H₆O)_{b}H

Poloxamers and reverse poloxamers have terminal hydroxyl groups that can be functionalized. An example of a terminal functionalized poloxamer is poloxamer dimethacrylate (Pluronic F-127 dimethacrylate) as disclosed in U.S. Patent Publication No. 2003/0044468 to Cellesi et al. U.S. Pat. No. 6,517,933 discloses glycidyl-terminated copolymers of polyethylene glycol and polypropylene glycol.

Poloxamers and reverse poloxamers are surfactants with varying HLB values based upon the varying values of a and b, a representing the number of hydrophilic (polyethylene oxide) units (PEO) being present in the molecule and b representing the number of hydrophobic (polypropylene oxide) units (PPO) being present in the molecule. While poloxamers and reverse poloxamers are considered to be difunctional molecules (based on the terminal hydroxyl groups) they are also available in a tetrafunctional form known as poloxamines, trade name TETRONIC. For poloxamines, the molecules are tetrafunctional block copolymers terminating in primary hydroxyl groups and linked by a central diamine. Poloxamines have the following general structure:

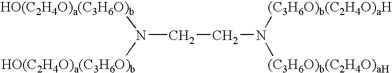

Reverse poloxamines are also known and have varying HLB values based upon the relative ratios of a to b.

Polyethers that are present at the surface of substrates have long been known to inhibit bacterial adhesion and to reduce the amount of lipid and protein deposition (non-fouling surface). In the present invention, we chemically modify poloxamer and poloxamine block copolymers and use them to coat medical devices having surface functional groups.

Medical devices such as ophthalmic lenses made from silicon containing materials have been investigated for a number of years. Such materials can generally be subdivided into two major classes, namely hydrogels and non-hydrogels. Non-hydrogels do not absorb appreciable amounts of water, whereas hydrogels can absorb and retain water in an equilibrium state. Regardless of their water content, both non-hydrogel and hydrogel silicon containing medical devices tend to have relatively hydrophobic, non-wettable surfaces that have a high affinity for lipids. This problem is of particular concern with contact lenses.

Those skilled in the art have long recognized the need for modifying the surface of such silicon containing contact lenses so that they are compatible with the eye. It is known that increased hydrophilicity of the contact lens surface improves the wettability of the contact lenses. This in turn is associated with improved wear comfort of contact lenses. Additionally, the surface of the lens can affect the lens's susceptibility to deposition, particularly the deposition of proteins and lipids from the tear fluid during lens wear. Accumulated deposition can cause eye discomfort or even inflammation. In the case of extended wear lenses (i.e. lenses used without daily removal of the lens before sleep), the surface is especially important, since extended wear lens should be designed for high standards of comfort and biocompatibility over an extended period of time.

Silicon containing lenses have been subjected to plasma surface treatment to improve their surface properties, e.g., surfaces have been rendered more hydrophilic, deposit resistant, scratch-resistant, or otherwise modified. Examples of previously disclosed plasma surface treatments include subjecting contact lens surfaces to a plasma comprising an inert gas or oxygen (see, for example, U.S. Pat. Nos. 4,055,378; 4,122,942; and 4,214,014); various hydrocarbon monomers (see, for example, U.S. Pat. No. 4,143,949); and combinations of oxidizing agents and hydrocarbons such as water and ethanol (see, for example, WO 95/04609 and U.S. Pat. No. 4,632,844). U.S. Pat. No. 4,312,575 to Peyman et al. discloses a process for providing a barrier coating on a silicon containing or polyurethane lens by subjecting the lens to an electrical glow discharge (plasma) process conducted by first subjecting the lens to a hydrocarbon atmosphere followed by subjecting the lens to oxygen during flow discharge, thereby increasing the hydrophilicity of the lens surface.

U.S. Pat. Nos. 4,168,112, 4,321,261 and 4,436,730, all issued to Ellis et al., disclose methods for treating a charged contact lens surface with an oppositely charged ionic polymer to form a polyelectrolyte complex on the lens surface that improves wettability.

U.S. Pat. Nos. 5,700,559 and 5,807,636, both to Sheu et al., discloses hydrophilic articles (for example, contact lenses) comprising a substrate, an ionic polymeric layer on the substrate and a disordered polyelectrolyte coating ionically bonded to the polymeric layer.

European Patent Application EP 0 963 761 A1 discloses biomedical devices with coatings that are said to be stable, hydrophilic and antimicrobial, and which are formed using a coupling agent to bond a carboxyl-containing hydrophilic coating to the surface by ester or amide linkages.

Polymerizable poloxamers and poloxamines as comonomers in forming polymeric devices have been developed and are disclosed in US pat. appln. Ser. No. 11/020,541, the content of which is incorporated by reference herein.

Because of the hydrophilic lipophilic balance (HLB) of these surfactants, the use of these materials as surface coatings for biomedical devices provides desirable results with regard to bacterial attachment and lipid deposition.

Surface structure and composition determine many of the physical properties and ultimate uses of solid materials including hydrogels. Characteristics such as wetting, friction, and adhesion or lubricity are largely influenced by surface characteristics. The alteration of surface characteristics is of special significance in biotechnical applications where biocompatibility is of particular concern. Thus, it is desired to provide a silicon containing hydrogel contact lens with an optically clear, hydrophilic surface film that will not only exhibit improved wettability, but which will generally allow the use of a silicon containing hydrogel contact lens in the human eye for extended period of time. In the case of a silicon containing hydrogel lens for extended wear, it would be further desirable to provide an improved silicon-containing hydrogel contact lens with an optically clear surface film that will not only exhibit improved lipid and microbial behavior, but which will generally allow the use of a silicon-containing hydrogel contact lens in the human eye for an extended period of time. Such a surface treated lens would be comfortable to wear in actual use and would allow for the extended wear of the lens without irritation or other adverse effects to the cornea.

It would also be desirable to apply these surface enhancing coatings to implantable medical devices such as intraocular lens materials to reduce the attachment of lens epithelial cells to the implanted device and to reduce friction as the intraocular lens passes through an inserter into the eye.

### SUMMARY

In accordance with the present invention, the invention relates generally to reactive surfactants and compositions comprising the surfactants as covalently bound coatings used in the manufacture of medical devices. According to preferred embodiments, the present invention relates to surface coated ophthalmic lenses formed from one or more functionalized poloxamers or poloxamines having reactive functionality that is complimentary to surface functionality of the ophthalmic lens.

In yet another embodiment, the invention is directed toward surface treatment of a polymeric device. The surface treatment comprises the covalent bonding of terminal reactive functionalized surfactant(s) to the surface of a polymeric medical device substrate by reacting complementary reactive functionalities of the terminal reactive functionalized surfactant(s) with surface reactive functionalities in monomeric units along the polymeric substrate examples of which include contact lenses, intraocular lenses, vascular stents, phakic intraocular lenses, aphakic intraocular lenses, corneal implants, catheters, implants, and the like, comprising a surface made by such a method.

In yet a further embodiment the invention is directed toward a method of forming a surface modified medical device, the method comprising providing a medical device comprising a substrate material that is a polymerized bulk monomer mixture prepared by copolymerizing a monomer mixture wherein the polymerized monomer mixture does not contain a surface functionality; providing a surface functionality to at least one surface of the medical device in a vessel; providing a surface modifying agent comprising a terminal functionalized surfactant having functionalized reactivity that is complimentary to the at least one group providing surface functionality of the medical device; contacting the at least one surface having reactive functionality of the medical device with the surface modifying agent, and; subjecting the device surface and surface modifying agent to reaction conditions suitable for forming a covalent bond between the device surface and the surface modifying agent to form a surface modified medical device.

### BRIEF DESCRIPTION OF THE DRAWINGS

FIG. 1 is a description of the general process used for coating lenses with polyether diepoxides;

FIG. 2 is an X-ray photon spectroscopy spectra of SofLens® (Bausch & Lomb) reacted with poloxamer diepoxide;

FIG. 3 is an X-ray photon spectroscopy spectra of PureVision® (Bausch & Lomb) and fluorovynagel as disclosed in U.S. Pat. No. 6,891,010, the contents of which are incorporated by reference herein, coated with polyether and poloxamer diepoxides;

FIG. 4 is a tabular representation of contact Angles for Fluorovynagel lenses with various surface treatments;

FIG. 5 shows the XPS spectra of control lens, the posterior surface of two lenses and the anterior surface of two lenses;

FIG. 6 shows dynamic contact angle study of Boston ES RGP (Bausch & Lomb) material treated with various Pluronic Epoxides as well as non-functionalized Pluronic. The data shows that there is a reduction in advancing contact angle with the use of Pluronic F127-DE and Pluronic F38-DE (this being the most significant) and that when F38-OH is used in the surface treatment the Pluronic can be rinsed away from the surface regenerating the original surface (similar contact angles),

FIG. 7 shows Dynamic contact angle study of Boston XO RGP(Bausch & Lomb) material treated with various Pluronic Epoxides as well as non-functionalized Pluronic. Same trends are observed as with Boston ES.

FIG. 8 shows Static Contact Angle measurements of Boston ES and Boston XO RGP materials treated with various Pluronic Epoxides as well as non-functionalized Pluronic.

### DETAILED DESCRIPTION

It should be understood that the expression “at least one surface” is not to be limited to meaning “at least one complete surface”. Surface coverage does not have to be even or complete to be effective for surface functionality.

The method of the present invention is useful with biocompatible materials including both soft and rigid materials commonly used for ophthalmic lenses, including contact lenses. Useful substrate materials can include vinyl functionalized polydimethylsiloxanes, optionally substituted with fluorine groups, copolymerized with hydrophilic monomers as well as fluorinated methacrylates and methacrylate functionalized fluorinated polyethylene oxides copolymerized with hydrophilic monomers. The present invention relates generally to reactive surfactants and compositions comprising the surfactants as covalently bound coatings used in the manufacture of medical devices. In preferred embodiments, the present invention relates to surface coated ophthalmic lenses formed from one or more functionalized poloxamers or poloxamines having reactive functionality that is complimentary to surface functionality of the ophthalmic lens.

Examples of substrate materials useful in the present invention are taught in U.S. Pat. Nos. 5,908,906 to Künzler et al.; 5,714,557 to Künzler et al.; 5,710,302 to Künzler et al.; 5,708,094 to Lai et al.; 5,616,757 to Bambury et al.; 5,610,252 to Bambury et al.; 5,512,205 to Lai; 5,449,729 to Lai; 5,387,662 to Künzler et al.; 5,310,779 to Lai and 6,891,010 to Künzler et al.; which patents are incorporated by reference as if set forth at length herein.

The present invention contemplates the use of terminal functionalized copolymers for medical devices including both “hard” and “soft” contact lenses.

As disclosed above, the invention is applicable to a wide variety of materials. Hydrogels in general are a well-known class of materials that comprise hydrated, cross-linked polymeric systems containing water in an equilibrium state. Silicon containing hydrogels generally have a water content greater than about 5 weight percent and more commonly between about 10 to about 80 weight percent. Such materials are usually prepared by polymerizing a mixture containing at least one silicon containing monomer and at least one hydrophilic monomer. Typically, either the silicon containing monomer or the hydrophilic monomer functions as a crosslinking agent (a crosslinker being defined as a monomer having multiple polymerizable functionalities) or a separate crosslinker may be employed. Applicable silicon containing monomeric units for use in the formation of silicon containing hydrogels are well known in the art and numerous examples are provided in U.S. Pat. Nos. 4,136,250; 4,153,641; 4,740,533; 5,034,461; 5,070,215; 5,260,000; 5,310,779; and 5,358,995.

Examples of applicable silicon-containing monomeric units include bulky polysiloxanylalkyl (meth)acrylic monomers. An example of bulky polysiloxanylalkyl (meth)acrylic monomers are represented by the following Formula I: 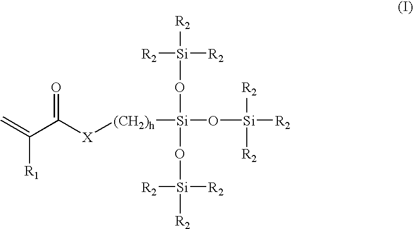
wherein:
X denotes —O— or —NR—;
each R₁ independently denotes hydrogen or methyl;
each R₂ independently denotes a lower alkyl radical, phenyl radical or a group 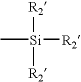
   wherein each R′₂ independently denotes a lower alkyl or phenyl radical; and h is 1 to 10.

Some preferred bulky monomers are methacryloxypropyl tris(trimethyl-siloxy)silane or tris(trimethylsiloxy)silylpropyl methacrylate, sometimes referred to as TRIS.

Another class of representative silicon-containing monomers includes silicon containing vinyl carbonate or vinyl carbamate monomers such as: 1,3-bis[4-vinyloxycarbonyloxy)but-1-yl]tetramethyl-disiloxane; 3-(trimethylsilyl)propyl vinyl carbonate; 3-(vinyloxycarbonylthio)propyl-[tris(trimethylsiloxy)silane]; 3-[tris(tri-methylsiloxy)silyl] propyl vinyl carbamate; 3-[tris(trimethylsiloxy)silyl] propyl allyl carbamate; 3-[tris(trimethylsiloxy)silyl]propyl vinyl carbonate; t-butyldimethylsiloxyethyl vinyl carbonate; trimethylsilylethyl vinyl carbonate; and trimethylsilylmethyl vinyl carbonate.

An example of silicon-containing vinyl carbonate or vinyl carbamate monomers are represented by Formula II: 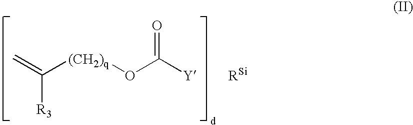
wherein:
Y′ denotes —O—, —S— or —NH—;
R^{Si} denotes a silicon containing organic radical;
R₃ denotes hydrogen or methyl;
d is 1, 2, 3 or 4; and q is 0 or 1.
Suitable silicon containing organic radicals R^{Si} include the following: 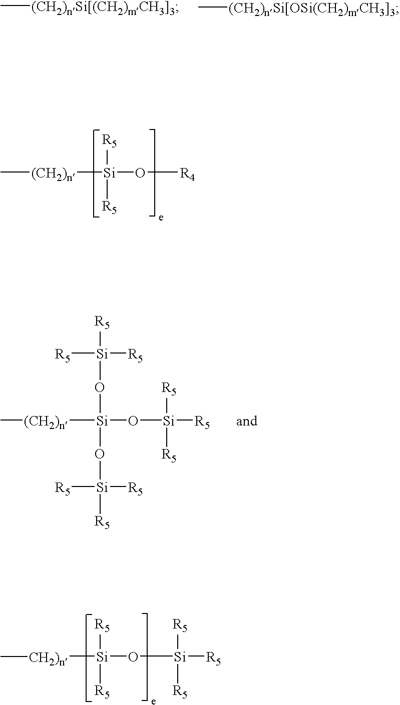
   wherein:
R₄ denotes 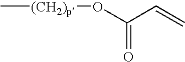
   wherein p′ is 1 to 6;
R₅ denotes an alkyl radical or a fluoroalkyl radical having 1 to 6 carbon atoms;
e is 1 to 200; n′ is 1, 2, 3 or 4; and m′ is 0, 1, 2, 3, 4 or 5.

An example of a particular species within Formula II is represented by Formula III.

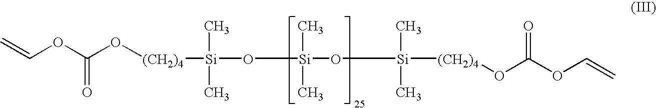

Another class of silicon-containing monomers includes polyurethane-polysiloxane macromonomers (also sometimes referred to as prepolymers), which may have hard-soft-hard blocks like traditional urethane elastomers. They may be end-capped with a hydrophilic monomer such as HEMA. Examples of such silicon containing urethanes are disclosed in a variety or publications, including Lai, Yu-Chin, “The Role of Bulky Polysiloxanylalkyl Methacryates in Polyurethane-Polysiloxane Hydrogels,” Journal of Applied Polymer Science, Vol. 60, 1193-1199 (1996). PCT Published Application No. WO 96/31792 discloses examples of such monomers, which disclosure is hereby incorporated by reference in its entirety. Further examples of silicon containing urethane monomers are represented by Formulae IV and V:
E(*D*A*D*G)ₐ*D*A*D*E′; or (IV)
E(*D*G*D*A)ₐ*D*G*D*E′; (V)
wherein:
D denotes an alkyl diradical, an alkyl cycloalkyl diradical, a cycloalkyl diradical, an aryl diradical or an alkylaryl diradical having 6 to 30 carbon atoms;
G denotes an alkyl diradical, a cycloalkyl diradical, an alkyl cycloalkyl diradical, an aryl diradical or an alkylaryl diradical having 1 to 40 carbon atoms and which may contain ether, thio or amine linkages in the main chain;
denotes a urethane or ureido linkage;
a is at least 1;
A denotes a divalent polymeric radical of Formula VI: 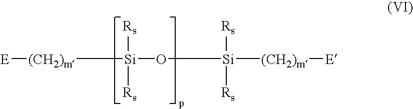
   wherein:
each R₅ independently denotes an alkyl or fluoro-substituted alkyl group having 1 to 10 carbon atoms which may contain ether linkages between carbon atoms;
m′ is at least 1; and
p is a number which provides a moiety weight of 400 to 10,000;
each of E and E′ independently denotes a polymerizable unsaturated organic radical represented by Formula VII: 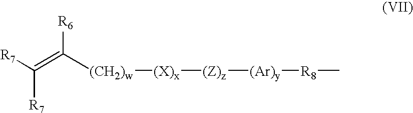
   wherein:
R₆ is hydrogen or methyl;
R₇ is hydrogen, an alkyl radical having 1 to 6 carbon atoms, or a —CO—Y—R₉ radical wherein Y is —O—, —S— or —NH—;
R₈ is a divalent alkylene radical having 1 to 10 carbon atoms;
R₉ is a alkyl radical having 1 to 12 carbon atoms;
X denotes —CO— or —OCO—;
Z denotes —O— or —NH—;
Ar denotes an aromatic radical having 6 to 30 carbon atoms;
w is 0 to 6; x is 0 or 1; y is 0 or 1; and z is 0 or 1.

A more specific example of a silicon containing urethane monomer is represented by Formula (VIII): 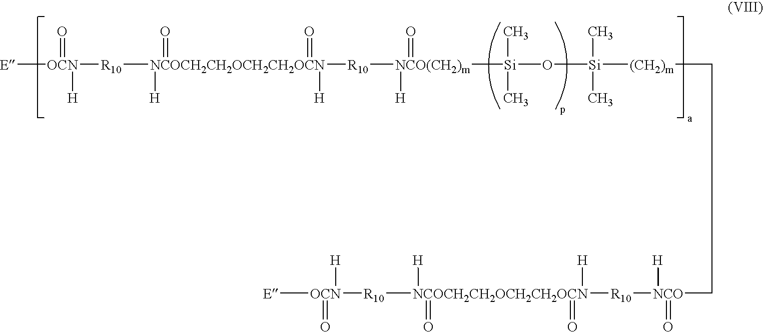
wherein m is at least 1 and is preferably 3 or 4, a is at least 1 and preferably is 1,
p is a number which provides a moiety weight of 400 to 10,000 and is preferably at least 30, R₁₀ is a diradical of a diisocyanate after removal of the isocyanate group, such as the diradical of isophorone diisocyanate, and each E″ is a group represented by:

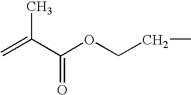

A preferred silicon containing hydrogel material comprises (in the bulk monomer mixture that is copolymerized) 5 to 50 percent, preferably 10 to 25, by weight of one or more silicon containing macromonomers, 5 to 75 percent, preferably 30 to 60 percent, by weight of one or more polysiloxanylalkyl (meth)acrylic monomers, and 10 to 50 percent, preferably 20 to 40 percent, by weight of a hydrophilic monomer. In general, the silicon containing macromonomer is a poly(organosiloxane) capped with an unsaturated group at two or more ends of the molecule. In addition to the end groups in the above structural formulas, U.S. Pat. No. 4,153,641 to Deichert et al. discloses additional unsaturated groups, including acryloxy or methacryloxy. Fumarate-containing materials such as those taught in U.S. Pat. Nos. 5,512,205; 5,449,729; and 5,310,779 to Lai are also useful substrates in accordance with the invention. Preferably, the silane macromonomer is a silicon-containing vinyl carbonate or vinyl carbamate or a polyurethane-polysiloxane having one or more hard-soft-hard blocks and end-capped with a hydrophilic monomer.

Suitable hydrophilic monomers comprise those monomers that, once polymerized, can form a complex with poly(acrylic acid). The suitable monomers form hydrogels, such as silicon-containing hydrogel materials useful in the present invention and comprise, for example, monomers that form complexes with poly(acrylic acid) and its derivatives. Examples of useful monomers include amides such as dimethylacrylamide, dimethylmethacrylamide, cyclic lactams such as n-vinyl-2-pyrrolidone and poly(alkene glycols) functionalized with polymerizable groups. Examples of useful functionalized poly(alkene glycols) include poly(diethylene glycols) of varying chain length containing monomethacrylate or dimethacrylate end caps. In a preferred embodiment, the poly(alkene glycol) polymer contains at least two alkene glycol monomeric units. Still further examples are the hydrophilic vinyl carbonate or vinyl carbamate monomers disclosed in U.S. Pat. Nos. 5,070,215, and the hydrophilic oxazolone monomers disclosed in U.S. Pat. No. 4,910,277. Other suitable hydrophilic monomers will be apparent to one skilled in the art.

Device Forming Additives and Comonomers

The monomer mix may, further as necessary and within limits not to impair the purpose and effect of the present invention, comprise various additives such as antioxidant, coloring agent, ultraviolet absorber and lubricant.

In the present invention, the monomer mix may be prepared by using, according to the end-use and the like of the resulting shaped polymer articles, one or at least two of the above comonomers and oligomers and, when occasions demand, one or more crosslinking agents.

Where the shaped polymer articles are for example medical products, in particular a contact lens, the monomer mix is suitably prepared from one or more of the silicon compounds, e.g. siloxanyl (meth)acrylate, siloxanyl (meth)acrylamide and silicon containing oligomers, to obtain contact lenses with high oxygen permeability.

The monomer mix of the present invention may include additional constituents such as crosslinking agents, internal wetting agents, hydrophilic monomeric units, toughening agents, and other constituents as is well known in the art.

Although not required, compositions within the scope of the present invention may include toughening agents, preferably in quantities of less than about 80 weight percent e.g. from about 5 to about 80 weight percent, and more typically from about 20 to about 60 weight percent. Examples of suitable toughening agents are described in U.S. Pat. No. 4,327,203. These agents include cycloalkyl acrylates or methacrylates, such as: methyl acrylate and methacrylate, t butylcyclohexyl methacrylate, isopropylcyclopentyl acrylate, t pentylcyclo-heptyl methacrylate, t butylcyclohexyl acrylate, isohexylcyclopentyl acrylate and methylisopentyl cyclooctyl acrylate. Additional examples of suitable toughening agents are described in U.S. Pat. No. 4,355,147. This reference describes polycyclic acrylates or methacrylates such as: isobomyl acrylate and methacrylate, dicyclopentadienyl acrylate and methacrylate, adamantyl acrylate and methacrylate, and isopinocamphyl acrylate and methacrylate. Further examples of toughening agents are provided in U.S. Pat. No. 5,270,418. This reference describes branched alkyl hydroxyl cycloalkyl acrylates, methacrylates, acrylamides and methacrylamides. Representative examples include: 4-t-butyl-2-hydroxycyclohexyl methacrylate (TBE); 4-t-butyl-2-hydroxycyclopentyl methacrylate; methacryloxyamino-4-t-butyl-2-hydroxycyclohexane; 6-isopentyl-3-hydroxycyclohexyl methacrylate; and methacryloxyamino-2-isohexyl-5 -hydroxycyclopentane.

In particular regard to contact lenses, the fluorination of certain monomers used in the formation of silicon containing hydrogels has been indicated to reduce the accumulation of deposits on contact lenses made therefrom, as described in U.S. Pat. Nos. 4,954,587, 5,079,319, 5,010,141 and 6,891,010. Moreover, the use of silicon containing monomers having certain fluorinated side groups, i.e. —(CF₂)—H, have been found to improve compatibility between the hydrophilic and silicon containing monomeric units, as described in U.S. Pat. Nos. 5,387,662 and 5,321,108.

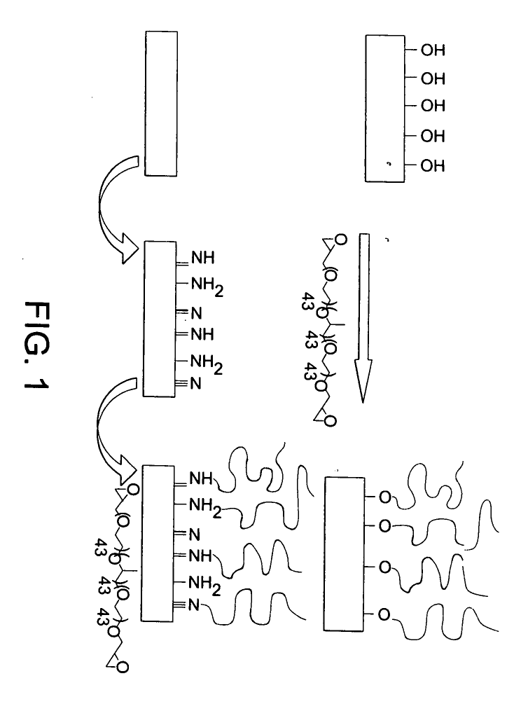

The present invention provides a method of surface modifying contact lenses and like medical devices through the use of complementary reactive functionality. Although only contact lenses will be referred to hereinafter for purposes of simplicity, such reference is not intended to be limiting since the subject method is suitable for surface modification of other medical devices such as phakic and aphakic intraocular lenses and corneal implants as well as contact lenses. As shown in FIG. 1A, surface reactive groups of the polymeric materials of the contact lenses and other biomedical devices are used to form covalent chemical linkages with the terminal reactive functionalized surfactant(s). The preferred terminal reactive functionalized surfactant(s) for use in the present invention are selected based on the specific reactive surface groups of the polymeric material to be coated. In accordance with the present invention, the one or more terminal reactive functionalized surfactant(s) selected for surface modification should have complementary chemical functionality to that of the surface reactive groups of the substrate. Such complementary chemical functionality enables a chemical reaction between the terminal reactive functionalized surfactant(s) and the surface reactive groups of the substrate to form covalent chemical linkages there between. The one or more terminal reactive functionalized surfactants are thus chemically bound to the surface of the surface reactive groups of the contact lens or like medical device to achieve surface modification thereof.

The poloxamer and/or poloxamine is functionalized to provide the desired reactivity at the terminal of the molecule. The functionality can be varied and is determined based upon the intended use of the functionalized PEO- and PPO-containing block copolymers. That is, the PEO- and PPO-containing block copolymers are reacted to provide terminal functionality that is complementary with the surface functionality of the device. By block copolymer we mean to define the poloxamer and/or poloxamine as having two or more blocks in their polymeric backbone(s). Variation in the number of PEO- and/or PPO- containing blocks in the copolymer will vary the HLB of the copolymer and thus its surface activity.

Selection of the functional group of the block copolymer is determined by the functional group of the reactive molecule on the surface of the device. For example, if the reactive molecule on the surface of the device contains a carboxylic acid group, a glycidyl group can be a reactive group of the reactive molecule. If the reactive molecule on the surface of the device contains hydroxy or amino functionality, the isocyanate group or carbonyl chloride can provide can be a reactive group of the reactive molecule. A wide variety of suitable combinations of functional groups of the reactive molecule complementary to reactive groups on the surface of the device will be apparent to those of ordinary skill in the art. For example, the terminal functional group of the terminal functionalized copolymer(s) may comprise a moiety selected from amine, hydroxyl, hydrazine, hydrazide, thiol (nucleophilic groups), carboxylic acid, carboxylic ester, including imide ester, orthoester, carbonate, isocyanate, isothiocyanate, aldehyde, ketone, thione, alkenyl, acrylate, methacrylate, acrylamide, sulfone, maleimide, disulfide, iodo, epoxy, sulfonate, thiosulfonate, silane, alkoxysilane, halosilane, and phosphoramidate. More specific examples of these groups include succinimidyl ester or carbonate, imidazolyl ester or carbonate, benzotriazole ester or carbonate, p-nitrophenyl carbonate, vinyl sulfone, chloroethylsulfone, vinylpyridine, pyridyl disulfide, iodoacetamide, glyoxal, dione, mesylate, tosylate, and tresylate. Also included are other activated carboxylic acid derivatives, as well as hydrates or protected derivatives of any of the above moieties (e.g. aldehyde hydrate, hemiacetal, acetal, ketone hydrate, hemiketal, ketal, thioketal, thioacetal). Preferred electrophilic groups include succinimidyl carbonate, succinimidyl ester, maleimide, benzotriazole carbonate, glycidyl ether, imidazoyl ester, p-nitrophenyl carbonate, acrylate, tresylate, aldehyde, and orthopyridyl disulfide.

The foregoing reaction sequences are intended to be illustrative, not limiting. Examples of reaction sequences by which PEO- and PPO-containing block copolymers can be functionalized to provide terminal reactive functionalized surfactant(s) are provided below:

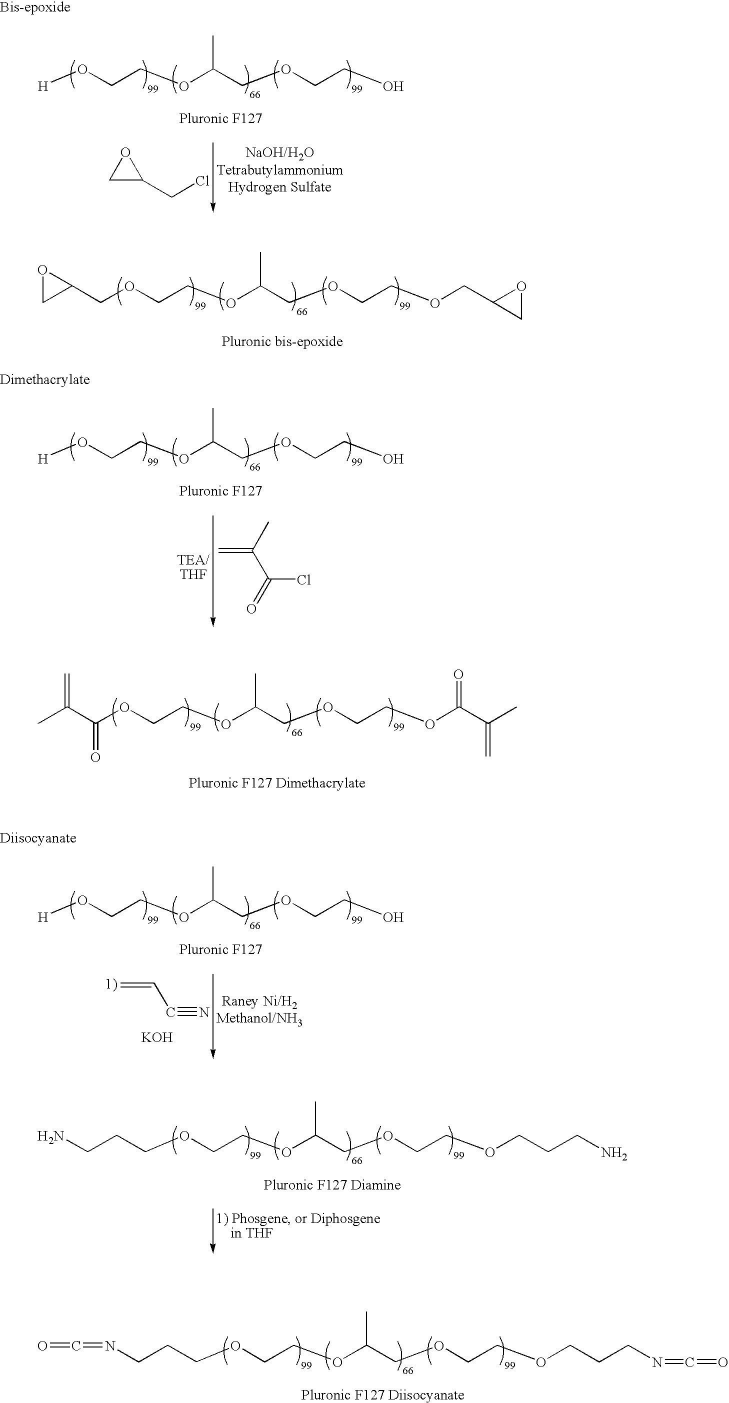

Further provided herein are certain exemplary, but non-limiting, examples of reactions for providing functionalized termini for PEO- and PPO-containing block copolymers. It is to be understood that one of ordinary skill in the art would be able to determine other reaction methods without engaging in an undue amount of experimentation. It should also be understood that any particular block copolymer molecule shown is only one chain length of a polydispersed population of the referenced material. Poloxamer block copolymers are known compounds and are generally available under the trademark PLURONIC. Poloxamers generally have the following structure:
HO(C₂H₄O)ₐ(C₃H₆O)_{b}(C₂H₄O)ₐH

Reverse poloxamers are also known block copolymers and generally have the following structure:
HO(C₃H₆O)_{b}(C₂H₄O)ₐ(C₃H₆O)_{b}H

PEO- and PPO-containing block copolymers are presently preferred. One such copolymer that can be used with the method of the invention, is Pluronic® F127, a block copolymer having the structure [(polyethylene oxide)₉₉-(polypropylene oxide)₆₆-(polyethylene oxide)g₉₉]. The terminal hydroxyl groups of the copolymer are functionalized to allow for the reaction of the copolymer with surface reactive groups of the polymeric substrate device.

As set forth above, for surface modification of contact lenses in accordance with the present invention, complementary reactive functionality is incorporated between the reactive surface groups of the contact lens material (i.e., the substrate) and the terminal reactive functionalized surfactant used as a surface modification treatment polymer (surface modifying agent). For example, if a surface modifying agent has epoxide functionality, then the contact lens material to be treated must have a residue with complementary functionality that will react with that of the surface modifying agent. In such a case, the contact lens material could include a reactive prepolymer such as bis-α,ω-fumaryl butyl polydimethyl siloxane, diacid to react with the surface modifying agent epoxide functionality. Likewise, if a contact lens is formed from material having a residue providing epoxide reactive, a surface modifying agent containing a 2-hydroxyethyl methacrylate terminal could be used for surface modification in accordance with the present invention. Such complementary chemical functionality enables a chemical reaction to occur between the surface reactive groups of the contact lens and the functional groups of the one or more surface modifying agent's. This chemical reaction between functional groups forms covalent chemical linkages there between. For example, a contact lens containing prepolymer having surface hydroxyl functional groups would preferably undergo surface modification using surface modifying agent's containing carboxylic acid functional groups, isocyanate functional groups or epoxy functional groups. Likewise, a contact lens containing prepolymer having surface carboxylic acid groups would preferably undergo surface modification using reactive, hydrophilic surface modifying agent's containing glycidyl methacrylate (GMA) monomer units to provide epoxy functional groups. The reaction of the contact lens containing surface reactive functional groups and the reactive surface modifying agent's is conducted under conditions known to those of skill in the art.

Examples of complementary functionality are provided below in Table 1.

TABLE 1

| RESIDUE HAVING A REACTIVE | COMPLEMENTARY |
|---|---|
| FUNCTIONAL GROUP | FUNCTIONALITY |
| Carboxylic acid, isocyanate, epoxy, | Alcohol, amine, thiol, epoxy |
| anhydride, lactone, lactam, oxazolone, | |
| maleimide, anhydride, acrylates | |
| Amine, thiol, alcohol | Carboxylic acid, isocyanate, |
| | epoxy, anhydride, lactone, |
| | lactam, oxazolone, maleimide, |
| | anhydride, acrylates |

More specifically, surface modification of contact lenses having reactive copolymers in accordance with the present invention may comprise one or more surface modifying agent's (surface modifying treatment polymer). Examples of surface modifying agent's useful in the practice of the present invention are terminal functionalized poloxamers and poloxamines.

Because of the hydrophilic lipophilic balance (HLB) of these surfactants, the use of these materials as surface coatings for biomedical devices provides desirable results with regard to bacterial attachment and lipid deposition.

As stated above, surface structure and composition determine many of the physical properties and ultimate uses of solid materials. Characteristics such as wetting, friction, and adhesion or lubricity are largely influenced by surface characteristics. The alteration of surface characteristics is of special significance in biotechnical applications where biocompatibility is of particular concern. Thus, it is desired to provide a silicon containing hydrogel contact lens with an optically clear, hydrophilic surface film that will not only exhibit improved wettability, but which will generally allow the use of a silicon containing hydrogel contact lens in the human eye for extended period of time. In the case of a silicon containing hydrogel lens for extended wear, it would be further desirable to provide an improved silicon-containing hydrogel contact lens with an optically clear surface film that will not only exhibit improved lipid and microbial behavior, but which will generally allow the use of a silicon-containing hydrogel contact lens in the human eye for an extended period of time. Such a surface treated lens would be comfortable to wear in actual use and would allow for the extended wear of the lens without irritation or other adverse effects to the cornea.

It would also be desirable to apply these surface enhancing coatings to implantable medical devices such as intraocular lens materials to reduce the attachment of lens epithelial cells to the implanted device and to reduce friction as the intraocular lens passes through an inserter into the eye.

The present invention is also directed toward surface treatment of a polymeric device. The surface treatment comprises the covalent bonding of terminal reactive functionalized surfactant(s) to the surface of a polymeric medical device substrate by reacting complementary reactive functionalities of the terminal reactive functionalized surfactant(s) with surface reactive functionalities in monomeric units along the polymeric substrate.

In the case where reactive groups are not present in the substrate material, they can be added using a surface activation treatment such as oxygen plasma, ammonia-butadiene-ammonia (ABA) treatments and hydrogen-ammonia-butadiene-ammonia (HABA) treatments (shown in FIG. 1B). Plasma treatment of substrate materials is known and is described in U.S. Pat. Nos. 6,193,369 Valint et al., 6,213,604 Valint et al. and 6,550,915 Grobe, III, the contents of each being incorporated by reference herein.

The process conditions of the present invention may be substantially the same as those in conventional plasma polymerization. The degree of vacuum during plasma polymerization may be 1×10⁻³ to 1 torr and the flow rate of the gas flowing into the reactor may be, for example, 0.1 to 300 cc (STP)/min in the case of the reactor having an inner volume of about 100 liter. The above-mentioned hydrogen gas may be mixed with an inert gas such as argon, helium, xenon, neon or the like before or after being charged into the reactor. The addition of halogenated alkanes is unnecessary but not deleterious, and may be present in combination with the hydrogen, preferably at an atomic ratio of less than ten percent of gaseous halogen to hydrogen. The substrate temperature during plasma polymerization is not particularly limited, but is preferably between 0° and 300° C.

The type of discharge to be used for the generation of plasma is not particularly limited and may involve the use of DC discharge, low frequency discharge, high frequency discharge, corona discharge or microwave discharge. Also, the reaction device to be used for the plasma polymerization is not particularly limited. Therefore either an internal electrode system or an electrodeless system may be utilized. There is also no limitation with respect to the shape of the electrodes or coil, or to the structure or the cavity or antenna in the case of microwave discharge. Any suitable device for plasma polymerization, including known or conventional devices, can be utilized.

Preferably, the plasma is produced by passing an electrical discharge, usually at radio frequency, through a gas at low pressure (0.005-5.0 torr). Accordingly, the applied radio frequency power is absorbed by atoms and molecules in the gaseous state, and a circulating electrical field causes these excited atoms and molecules to collide with one another as well as the walls of the chamber and the surface of the material being treated. Electrical discharges produce ultraviolet (UV) radiation, in addition to energetic electrons and ions, atoms (ground and excited states), molecules and radicals. Thus, a plasma is a complex mixture of atoms and molecules in both ground and excited states which reach a steady state after the discharge is begun.

The effects of changing pressure and discharge power on the plasma treatment is generally known to the skilled artisan. The rate constant for plasma modification generally decreases as the pressure is increased. Thus, as pressure increases the value of E/P, the ratio of the electric field strength sustaining the plasma to the gas pressure, decreases and causes a decrease in the average electron energy. The decrease in electron energy in turn causes a reduction in the rate coefficient of all electron-molecule collision processes. A further consequence of an increase in pressure is a decrease in electron density. Taken together, the effect of an increase in pressure is to cause the rate coefficient to decrease. Providing that the pressure is held constant there should be a linear relationship between electron density and power. Thus, the rate coefficient should increase linearly with power.

In one embodiment of the invention, a hydrogen-plasma treated fluorinated polymeric surface is subsequently oxidized by an oxidizing plasma, e.g., employing O₂ (oxygen gas), water, hydrogen peroxide, air, ammonia, etc., or mixtures thereof, creating radicals and oxidized functional groups. Such oxidation can render the surface of a lens more reactive. Further surface treatment can then be carried out, for example, the attachment of surface modifying agents.

In practice, contact lenses may be surface treated, for example, by placing them, in their unhydrated state, within an electric glow discharge reaction vessel (e.g., a vacuum chamber). Such reaction vessels are commercially available. The lenses may be supported within the vessel on an aluminum tray (which acts as an electrode) or with other support devices designed to adjust the position of the lenses. The use of specialized support devices which permit the surface treatment of both sides of a lens are known in the art and may be used in the present invention.

The plasma treatment, for example hydrogen or hydrogen in an inert gas such as argon, may suitably utilize an electric discharge frequency of, for example, 13.56 MHz, suitably between about 100-1000 watts, preferably 200 to 800 watts, more preferably 300 to 500 watts, at a pressure of about 0.1-1.0 torr. The plasma-treatment time is preferably at least 2 minutes total, and most preferred at least 5 minutes total. Optionally, the lens may be flipped over to better treat both sides of the lens. The plasma-treatment gas is suitably provided at a flow rate of 50 to 500 sccm (standard cubic centimeters per minute), more preferably 100 to 300 sccm. The thickness of the surface treatment is sensitive to plasma flow rate, chamber temperature, chamber loading of samples and sample holders (trays) or other variables as will be understood by the skilled artisan. Since the coating is dependent on a number of variables, the optimal variables for obtaining the desired or optimal coating may require some adjustment. If one parameter is adjusted, a compensatory adjustment of one or more other parameters may be appropriate, so that some routine trial and error experiments and iterations thereof may be necessary in order to achieve the coating according to the present invention. However, such adjustment of process parameters, in light of the present disclosure and the state of the art in plasma treatment, should not involve undue experimentation. As indicated above, general relationships among process parameters are known by the skilled artisan, and the art of plasma treatment has become well developed in recent years. Others methods of surface treatment, known to those skilled in the art, include but are not limited to, atmospheric plasma, corona and UV/ozone treatment

Methods of coating the substrate would include dip coating of the substrate into a solution containing the surface modifying agent. The solution containing the surface modifying agent may contain substantially the surface modifying agent in solvent or may contain other materials such as cleaning and extracting materials. Other methods could include spray coating the device with the surface modifying agent. In order for the covalent bonding reaction to occur, it may be necessary to use suitable catalysts, for example, condensation catalyst. Alternatively, the substrate and the other surface modifying agent may be subjected to autoclave conditions. In certain embodiments, the substrate and the surface modifying agent may be autoclaved in the packaging material that will contain the coated substrate. Once the reaction between the substrate and the surface modifying agent has occurred, the remaining surface modifying agent could be substantially removed and packaging solution would be added to the substrate packaging material. Sealing and other processing steps would then proceed as they usually do.

The terminal reactive functionalized surfactant(s) useful in certain embodiments of the present invention may be prepared according to syntheses well known in the art and according to the methods disclosed in the following examples. Surface modification of contact lenses using one or more surface modifying agents in accordance with the present invention is described in still greater detail in the examples that follow.

### EXAMPLES

### Example 1

### Synthesis of Terminal Functionalized Copolymers

6.00 g of PLURONIC F127 was placed in a round bottom flask and dried thoroughly via azeotropic distillation of toluene (100 ml). The round bottom flask was then fitted with a reflux condenser and the reaction was blanketed with Nitrogen gas. Anhydrous tetrahydrofuran (THF) (60 ml) was added to the flask and the reaction was chilled to 5° C. and 15 equivalents (based upon the hydroxyl groups) of triethylamine (TEA) was added (2.0 ml). 1.4 ml of methacryoyl chloride (15 equivalents) was dropped into the reaction mixture through an addition funnel and the reaction mixture was allowed to warm to room temperature and then stirred overnight. The reaction mixture was then heated to 65° C. for 3 hours. Precipitated salt (TEA-HCl) was filtered from the reaction mixture and the filtrate was concentrated to a volume of around 355 mL and precipitated into cold heptane. Two further reprecipitations were performed to reduce the amount of TEA-HCl salt to less than 0.2% by weight. NMR analysis of the final polymer showed greater than 90% conversion of the hydroxyl groups to the methacrylated groups.

### Example 2

### Synthesis of Surfactant Epoxides

10.00 gms of PLURONIC F38 (2.13E-03 mol) are placed in a round bottom flask and dried thoroughly via azeotropic distillation of toluene and then dissolved in 100 mL of THF. 10 equivalents of solid NaH were added into the flask (0.51 gm; 2.13E-02 mol). Next 1.67 mL of epichlorohydrin (2.13E-03 mol) was added to the reaction mixture and mixed well and the reaction mixture was heated to reflux for 24 hours. The reaction mixture was cooled and a scoop of magnesium sulfate and silica gel was added. Mixed well for 5 minutes and then filtered off the insolubles. Filtrate was concentrated to around 30 mL final volume and the product was precipitated into heptane and isolated by filtration. NMR confirms the presence of epoxide groups on the termini of the polymer.

### Example 3

### Purification of Terminal Functionalized Copolymers

Different dimethacrylated PLURONICS (BASF) and TETRONICS (BASF) had to be purified by different techniques depending upon their ability to precipitate and their solubility in water. The purification technique used for each example is listed in the table 2 below:

TABLE 2

| # | | Mol. Wt. | % EO/HLB | Form | Method | Water Soluble |
|---|---|---|---|---|---|---|
| | Pluronics | | | | | |
| 1 | Pluronic F127 | 12,600 | 70/22 | solid | Prec/Dialysis | + |
| 2 | Pluronic P105 | 6,500 | 50/15 | paste | Dialysis | + |
| 3 | Pluronic P123 | 5,750 | 30/8 | paste | Dialysis | + |
| 4 | Pluronic F38 | 4,700 | 80/31 | solid | Prec/Dialysis | + |
| 5 | Pluronic L101 | 3,800 | 10/1 | liquid | Water/Centrifuge | − |
| 6 | Pluronic L121 | 4,400 | 10/1 | liquid | Water/Centrifuge | − |
| | Reverse Pluronics | | | | | |
| 7 | Pluronic 10R5 | 1,950 | 50/15 | liquid | Dialysis | + |
| 8 | Pluronic 31R1 | 3,250 | 10/1 | liquid | Water/Centrifuge | − |
| 9 | Pluronic 25R4 | 3,600 | 40/8 | paste | Dialysis | + |
| | Tetronics | | | | | |
| 10 | Tetronic 1107 | 15,000 | 70/24 | solid | Prec/Dialysis | + |
| 11 | Tetronic 904 | 6,700 | 40/15 | paste | Dialysis | + |
| 12 | Tetronic 908 | 25,000 | 80/31 | solid | Prec/Dialysis | + |
| 13 | Tetronic 1301 | 6,800 | 10/2 | liquid | Water/Centrifuge | − |
| | Reverse Tetronics | | | | | |
| 14 | Tetronic 150R1 | 8,000 | 10/1 | liquid | Water/Centrifuge | − |
| 15 | Tetronic 90R4 | 7,240 | 40/7 | liquid | Dialysis | + |
| | Other | | | | | |
| 16 | PEO | 10,000 | 100/>31 | solid | Prec/Dialysis | + |
| 17 | PPO | 3,500 | 0/<1 | liquid | Water/Centrifuge | − |

KEY: The method column refers to the method that can be used for purification of the resulting functionalized surfactant. Prec means that the polymer can be dissolved into Tetrahydrofuran (THF) and precipitated in hexane, with several reprecipitations leading to pure product (3x). Dialysis of the water soluble functionalized surfactant in 500-1000 molecular weight cut off dialysis tubing followed by freeze drying is a viable technique for purification of all water soluble PLURONICS and TETRONICS. Centrifuge means that functionalized surfactant is stirred in water and the water insoluble functionalized surfactant is then isolated by centrifugation and decanting off the top water layer. In the Water Soluble column, + means the functionalized surfactant is water-soluble and − means it is insoluble in water.

### Example 4

### Surface Coating of Contact Lenses

The general procedure for coating a contact lens is as follows:

1) Lenses were soaked in purified water to remove buffers.

2) Lenses were transferred into autoclave vials that contained 4 mL of a coating solution, i.e. surface modifying agent. Coating solutions were prepared by dissolving either 0.1% or 1.0% by weight of the epoxide functionalized Pluronics in pure water. As a control experiment, 3 weight % of the non-functionalized Pluronic was also included as a separate coating solution.

3) Lenses were autoclaved in the coating solutions for 30 minutes at 121 degrees centigrade.

4) After removal from the autoclave, the lenses were rinsed three times with purified water and placed back in autoclave vials with 3 mL of phosphate buffered saline (pH=7.4). The lenses were then reautoclaved. The lenses could now be submitted for surface analysis.

### Example 5

### Analysis of Coated Lenses by XPS:

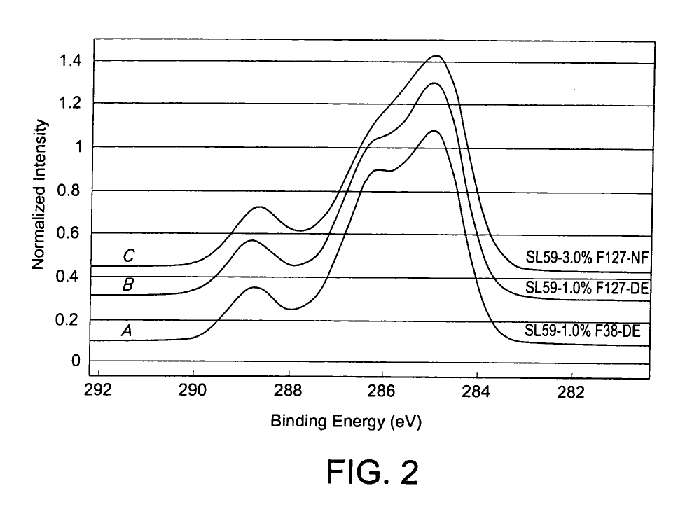

SofLens® 59 (Bausch & Lomb Incorporated) lenses that were coated with Pluronic diepoxides were examined using X-ray photoelectron spectroscopy (XPS) and the results are shown in FIG. 2. As shown in the figure, when the lenses were autoclaved with 1.0% F38-DE and 1.0% F127-DE (Curves A and B), the presence of the Pluronic coating is demonstrated by the broadening of the Cls peak in the XPS spectra due to the increased contribution of C—O. As a control experiment, when the lenses are autoclaved with 3.0% F127-NF (non-functionalized Pluronic F127 has two hydroxy termini) the XPS spectra (curve C) does not have the shoulder in the Cls peak region and this correlates well with the spectra of the original uncoated lens material (curve not shown).

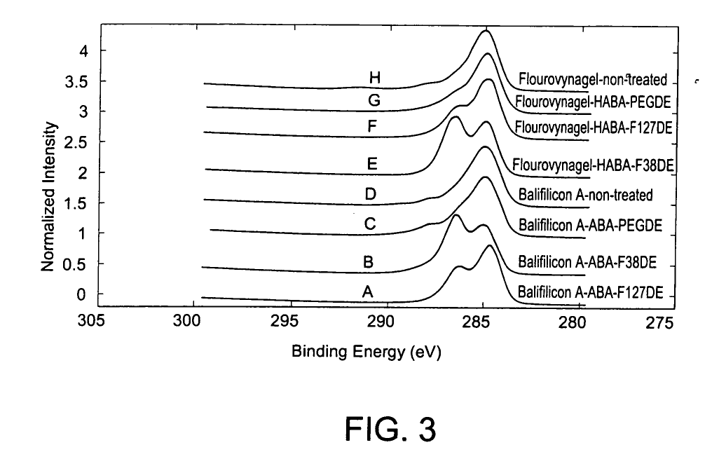

Both PureVision® (balafilicon A, Bausch & Lomb Incorporated) and Fluorovynagel lenses were also coated with polyether diepoxides (F127-DE; F38-DE; and PEG-DE) and their surfaces were studied with XPS. The results are shown in FIG. 3. When ABA treated PureVision® (Bausch & Lomb Incorporated) was autoclaved with 1% F127-DE and 1% F38-DE (curves A and B), the presence of the Pluronic coating is demonstrated by the broadening of the Cls peak in the XPS spectra due to the increased contribution of C—O (pronounced shoulder on the left side of the peak). Interestingly, when the lenses are autoclaved with PEG-DE (curve C), the Cls peak looks more like the non-treated lens surface (curve D) indicating that the PEG-DE does not coat the lens surface as efficiently as the Pluronic diepoxides, most likely due to the surface activity of the Pluronics. For HABA treated Fluorovynagel, autoclave coating with 1% F38-DE and 1% F127-DE (curves E and F) shows the presence of the Pluronic coatings with the pronounced shoulder on the left side of the Cls peak. Again the PEG-DE coated lenses look more similar to the untreated lenses showing that less of the polyether is present on the surface.

### Example 6

### Contact Angle Analysis

Description of Samples:

Contact angle analysis was performed on 12 lots of lenses listed below treated in the following manner:
JGL2454-046A: Fluorovynagel—untreated
JGL2454-046B: Fluorovynagel—HABA Plasma and 1.0% F127-DE
JGL2454-046C: Fluorovynagel—HABA Plasma and 1.0% F38-DE
JGL2454-046D: Fluorovynagel—HABA Plasma and 1.0% PEG-DE
JGL2454-046E: Fluorovynagel—HABA Plasma only
JGL2454-046F: Fluorovynagel—Oxygen Plasma and 1.0% F127-DE
JGL2454-046G: balafilicon A—untreated
JGL2454-046H: balafilicon A—ABA Plasma and 1.0% F127-DE
JGL2454-046I: balafilicon A—ABA Plasma and 1.0% F38-DE
JGL2454-046J: balafilicon A—No Plasma and 1.0% F127-DE
JGL2454-046K: balafilicon A—Oxygen Plasma
JGL2454-046L: balafilicon A—ABA Plasma

The analytical methods applied to fluorovynagels are the same as those applied to balafilcon A lenses.

Methods and Materials:

The instrument used for measurement was an AST Products Video Contact Angle System (VCA) 2500XE. This instrument utilizes a low-power microscope that produces a sharply defined image of the water drop, which is captured immediately on the computer screen.

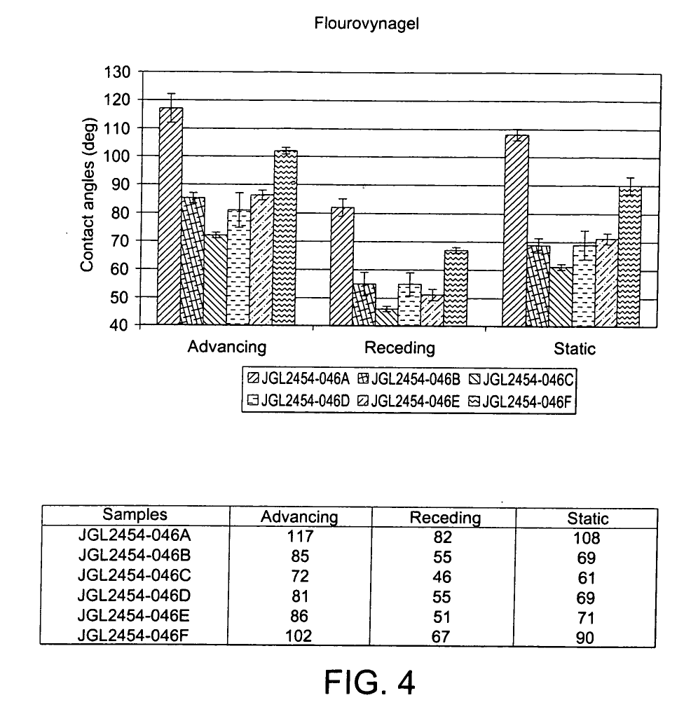

Surface Tension of the water used for analysis was measured by the dynamic contact angle method and recorded as 73.3 dynes/cm prior to testing. A 0.8 μl drop is dispensed from the syringe and the sample is moved upward until it is in contact with the droplet. After contact, an image was captured and analyzed to obtain the advancing contact angle. Immediately after capturing the advancing image, the stage was slowly lowered until the drop of water was near losing contact with the end of the syringe, an image was captured and used for analysis to obtain the receding contact angle. The drop was then allowed to lose contact with the syringe tip and sit on the surface, this image was captured and used to obtain the static contact angle. The contact angle is calculated by placing five markers along the circumference of the drop . The software calculates a curve representing the circumference of the drop and the contact angle is recorded. The contact angle (Θ) is shown in FIG. 4. Both a right and left contact angle are reported for each measurement.

In FIG. 4 and the table below it can be seen that all of the surface treatments produced a more wettable surface than the untreated lenses in the case of Fluorovynagel (lower contact angles). In particular, the F38-DE showed the lowest contact angle which can be consistent with the fact that it is 80% hydrophile. It can also be noted that the oxygen plasma is not as effective as the HABA plasma in providing a hydrophilic surface.

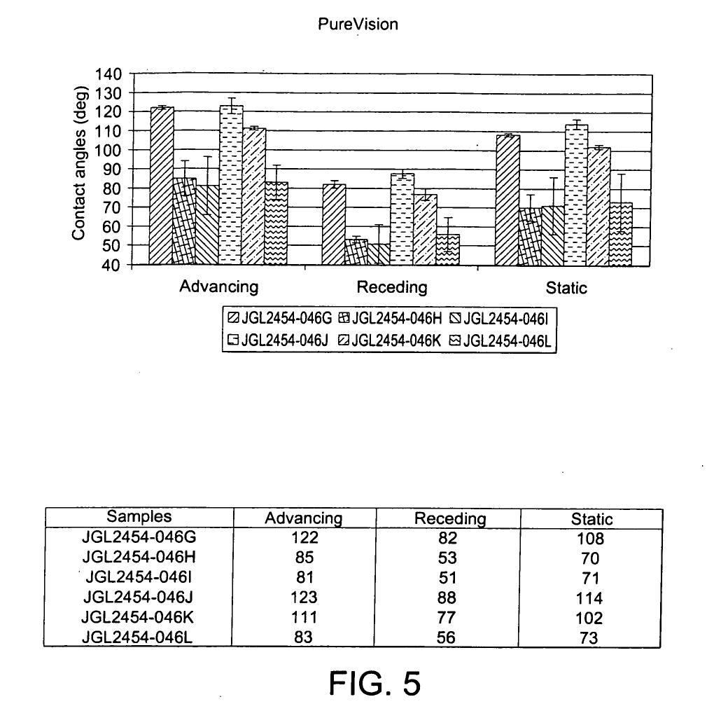

In FIG. 5 and table below it can be seen that the ABA plasma treatments followed by polymer coatings provided a more hydrophilic surface than unmodified PureVision. Again it can also be seen that the oxidative plasma is not as good as the ABA plasma in producing a wettable surface. Furthermore, without plasma surface treatment there is little to no polymer binding to the surface.

### Example 7

### Bacterial Attachment Assay

Method:

Three lenses from each set were soaked in 2 mL suspensions of ˜1×10⁴ of a clinical isolate of Pseudomonas aeruginosa GSU#3 in PBS for two hours at 30-35° C. in rotary shaking. The lenses were then removed from the suspension and rinsed gently in PBS to remove non-adherent bacterial cells. Lenses were plated in growth medium and the number of recovered colony forming units (CFU) was determined after two days growth at 30-35° C. The percent reduction in number of attached, viable and recovered bacterial cells were calculated relative to the average number of CFUs recovered from control contact lenses (untreated contact lenses).

Results:

The results of the bacterial attachment study are presented in table 3. In general the plasma treated and polymer coated lenses had a smaller number of recovered CFUs when compared with the untreated control lenses (in bold face in the table below). In both data sets the lens surface coated with Pluronic F38-DE showed the highest degree of inhibition in bacterial (Pseudomonas) attachment.

TABLE 3
The inhibition of Pseudomonas aeruginosa GSU#3 attachment to surface
treated Fluorovynagel and PureVision ® lenses.

| | Recovered | | | | |
|---|---|---|---|---|---|
| | Pa | GSU | CFU | AVG | % Reduction |
| Surface Treated Fluorovynagel | | | | | |
| Untreated Control Lens FV (A) | 50 | 53 | 61 | 55 | 0 |
| HABA Plasma and 1.0% F127-DE (B) | 34 | 45 | 25 | 35 | 37 |
| HABA Plasma and 1.0% F38-DE (C) | 23 | 28 | 32 | 28 | 50 |
| HABA Plasma and 1.0% PEG-DE (D) | 33 | 44 | 43 | 40 | 27 |
| HABA Plasma only (E) | 18 | 23 | 31 | 24 | 56 |
| Oxygen Plasma and 1.0% F127-DE | 45 | 41 | 52 | 46 | 16 |
| (F) | | | | | |
| Surface Treated PureVision | | | | | |
| Untreated Control Lens PV(G) | 49 | 51 | 54 | 51 | 0 |
| ABA Plasma and 1.0% F127-DE (H) | 24 | 36 | 38 | 33 | 36 |
| ABA Plasma and 1.0% F38-DE (I) | 22 | 21 | 23 | 22 | 57 |
| No Plasma and 1.0% F127-DE (J) | 27 | 37 | 31 | 32 | 38 |
| Oxygen Plasma (K) | 35 | 41 | 29 | 35 | 31 |
| ABA Plasma (L) | 40 | 36 | 35 | 37 | 27 |

### Example 8

### Coating of RGP Contact Lenses

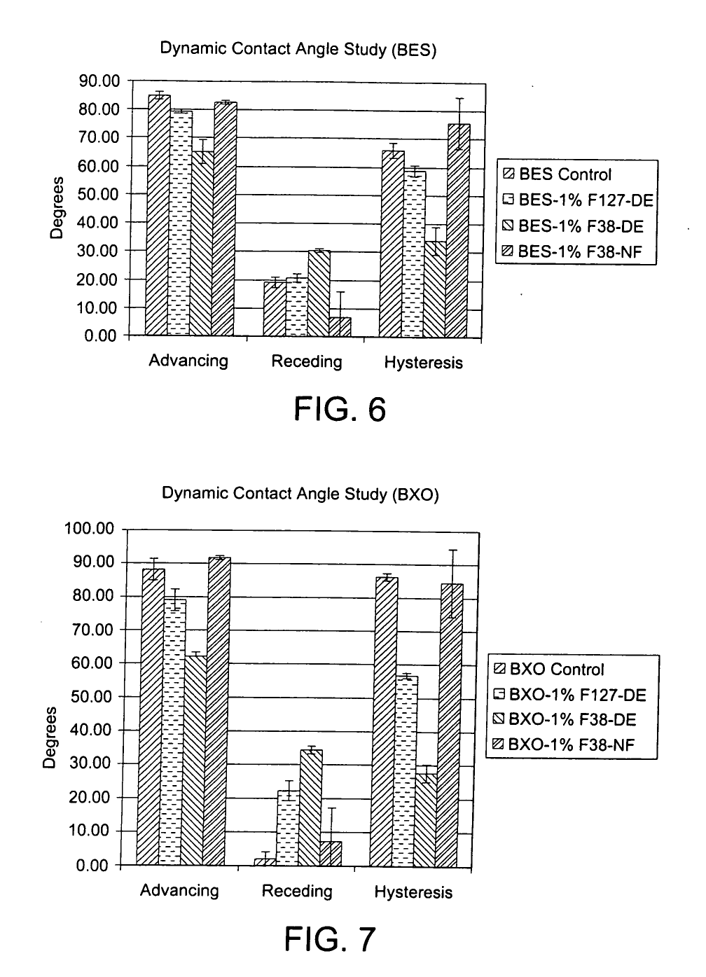

Flat substrates made from Boston ES (Bausch & Lomb Incorporated) and Boston XO (Bausch & Lomb Incorporated) were thoroughly cleaned with lens cleaner and rinsed with deionized water. These substrates were then transferred to vials containing coating solutions. Coating solutions contained 1% by weight of Pluronic diepoxide and 0.2% of methyldiethanolamine. Flats were placed in 3 mL of coating solution and placed in an oven at 55 degrees centigrade for 8 hours and then removed and thoroughly rinsed with deionized water. Both dynamic and static contact angle measurements were performed. For DCA (dynamic contact angle) measurements, rectangular wafers were prepared for the various substrates and the advancing and receding contact angles were determined for each substrate by sequentially inserting and withdrawing the samples in phosphate buffered saline (PBS) at room temperature using the Wilhelmy Plate technique. (FIGS. 6 and 7) Static contact angles were obtained using the AST Video Contact Angle System (VCA) 2500XE. A 0.8 μl drop of water is dispensed from a syringe onto the substrate and the image is immediately captured and analyzed using the VCA software.

Dynamic contact angle study of Boston ES RGP material treated with various Pluronic Epoxides as well as non-functionalized Pluronic is shown in FIG. 6. The data shows that there is a reduction in advancing contact angle with the use of Pluronic F127-DE and Pluronic F38-DE (this being the most significant) and that when F38-OH is used in the surface treatment the Pluronic can be rinsed away from the surface regenerating the original surface (similar contact angles).

Dynamic contact angle study of Boston XO RGP material treated with various Pluronic Epoxides as well as non-functionalized Pluronic. The same trends are observed as with Boston ES as Boston XO

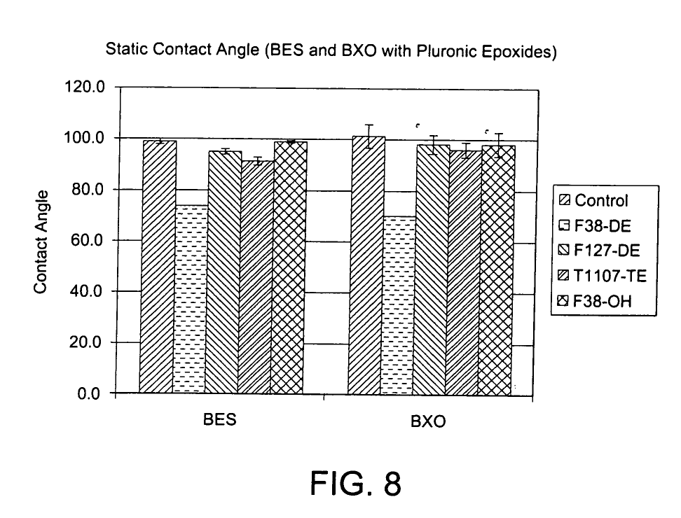

The Static Contact Angle measurements of Boston ES and Boston XO RGP materials treated with various Pluronic Epoxides as well as non-functionalized Pluronic is shown in FIG. 8. The data supports the results of the DCA measurements and shows that there is a significant reduction in contact angle with the use of Pluronic F38-DE for the surface treatment and a small decrease in contact angle when F127-DE or T1107-TE is used. In addition, non-functionalized Pluronic can be rinsed away from the surface regenerating the original surface (similar contact angles

Contact lenses manufactured using the unique materials of the present invention are used as customary in the field of ophthalmology. While there is shown and described herein certain specific structures and compositions of the present invention, it will be manifest to those skilled in the art that various modifications may be made without departing from the spirit and scope of the underlying inventive concept and that the same is not limited to particular structures herein shown and described except insofar as indicated by the scope of the appended claims.

What is claimed is:

1. A method of forming a surface modified medical device, the method comprising:
providing a medical device made of a copolymer that is the polymerization product of a monomer mixture comprising a monomer that has at least one group providing surface functionality to at least one surface of the medical device;
providing a surface modifying agent comprising a terminal functionalized surfactant having functionalized reactivity that is complimentary to the at least one group providing surface functionality of the medical device;
contacting the at least one surface having reactive functionality of the medical device with the surface modifying agent, and;
subjecting the device surface and surface modifying agent to reaction conditions suitable for forming a covalent bond between the device surface and the surface modifying agent to form a surface modified medical device.

2. The method of claim 1 wherein the monomer mixture comprises a silicon containing monomer.

3. The method of claim 2 wherein the silicon containing monomer comprises a silicon containing monomer selected from the group consisting of silicon containing vinyl carbonates, silicon containing vinyl carbamates, polyurethane-polysiloxanes having one or more hard-soft-hard blocks and end-capped with a hydrophilic monomer, fumarate containing silicon containing monomers, poly(organosiloxanes) capped with an unsaturated group at two or more ends of the molecule, polyurethane-polysiloxane macromonomers and mixtures thereof.

4. The method of claim 1 wherein the copolymer in the bulk monomer mixture that is to be copolymerized comprises 5 to 50 percent by weight of one or more silicon containing macromonomers, 5 to 75 percent by weight of one or more polysiloxanylalkyl (meth)acrylic monomers, and 10 to 50 percent by weight of a hydrophilic monomer.

5. The method of claim 1 wherein the copolymer in the bulk monomer mixture that is to be copolymerized comprises 10 to 25 percent by weight of one or more silicon containing macromonomers, 30 to 60 percent by weight of one or more polysiloxanylalkyl (meth)acrylic monomers, and 20 to 40 percent by weight of a hydrophilic monomer.

6. The method of claim 1 wherein the medical device comprises hydrogel materials.

7. The method of claim 1 wherein the medical device comprises silicon containing hydrogel materials.

8. The method of claim 1 wherein the medical device comprises vinyl functionalized polydimethylsiloxanes copolymerized with hydrophilic monomers.

9. The method of claim 1 wherein the medical device comprises fluorinated monomers.

10. The method of claim 1 wherein the medical device comprises methacrylate functionalized fluorinated polyethylene oxides copolymerized with hydrophilic monomers.

11. The method of claim 1 wherein the medical device is selected from the group consisting of heart valves, intraocular lenses, contact lenses, intrauterine devices, vessel substitutes, artificial ureters, vascular stents, phakic intraocular lenses, aphakic intraocular lenses, corneal implants, catheters, implants, and artificial breast tissue.

12. The method of claim 11 wherein the medical device formed is a soft contact lens.

13. The method of claim 12 wherein the medical device is a silicon containing hydrogel contact lens material.

14. The method of claim 1 wherein the medical device has at least one surface functionality selected from the group consisting of epoxides, carboxylic acids, anhydrides, oxazolinones, lactams, lactones, amines, hydroxys, hydrazines, hydrazides, thiols, nucleophilic groups, electrophilic groups, carboxylic esters, imide esters, orthoesters, carbonates, isocyanates, isothiocyanates, aldehydes, ketones, thiones, alkenyls, acrylates, methacrylates, acrylamides, sulfones, maleimides, disulfides, iodos, sulfonates, thiosulfonates, silanes, alkoxysilanes, halosilanes, phosphoramidate and alcohol functionality.

15. The method of claim 14 wherein the surface functionality is selected from the group consisting of aldehyde hydrates, hemiacetals, acetals, ketone hydrates, hemiketals, ketals, thioketals, and thioacetals.

16. The method of claim 14 wherein the surface functionality is selected from the group consisting of succinimidyl carbonate, succinimidyl ester, maleimide, benzotriazole carbonate, glycidyl ether, imidazoyl ester, p-nitrophenyl carbonate, acrylate, tresylate, aldehyde, and orthopyridyl disulfide.

17. The method of claim 1 wherein the surface modifying agent has at least one functionalized terminal selected from the group consisting of epoxides, carboxylic acids, anhydrides, oxazolinones, lactams, lactones, amines, hydroxys, hydrazines, hydrazides, thiols, nucleophilic groups, electrophilic groups, carboxylic esters, imide esters, orthoesters, carbonates, isocyanates, isothiocyanates, aldehydes, ketones, thiones, alkenyls, acrylates, methacrylates, acrylamides, sulfones, maleimides, disulfides, iodos, sulfonates, thiosulfonates, silanes, alkoxysilanes, halosilanes, phosphoramidate and alcohol functionality.

18. The method of claim 17 wherein the surface modifying agent has at least one functionalized terminal selected from the group consisting of aldehyde hydrates, hemiacetals, acetals, ketone hydrates, hemiketals, ketals, thioketals, and thioacetals.

19. The method of claim 17 wherein the surface modifying agent has at least one functionalized terminal selected from the group consisting of succinimidyl carbonate, succinimidyl ester, maleimide, benzotriazole carbonate, glycidyl ether, imidazoyl ester, p-nitrophenyl carbonate, acrylate, tresylate, aldehyde, and orthopyridyl disulfide.

20. The method of claim 17 wherein the surface modifying agent has at least one functionalized terminal selected from the group consisting of succinimidyl carbonate, succinimidyl ester, maleimide, benzotriazole carbonate, glycidyl ether, imidazoyl ester, p-nitrophenyl carbonate, acrylate, tresylate, aldehyde, and orthopyridyl disulfide.

21. A method of forming a surface modified medical device, the method comprising:
providing a medical device made of a copolymer that is the polymerization product of a monomer mixture wherein the polymerized monomer mixture does not contain a surface functionality;
providing a surface functionality to at least one surface of the medical device;
providing a surface modifying agent comprising a terminal functionalized surfactant having functionalized reactivity that is complimentary to the at least one group providing surface functionality of the medical device;
contacting the at least one surface having reactive functionality of the medical device with the surface modifying agent, and;
subjecting the device surface and surface modifying agent to reaction conditions suitable for forming a covalent bond between the device surface and the surface modifying agent to form a surface modified medical device.

22. The method of claim 21 wherein the surface functionality is provided by a plasma treatment selected from the group consisting of gaseous plasma treatment, atmospheric plasma treatment, corona treatment and UV/ozone treatment.

23. The method of claim 22 wherein the gaseous plasma treatment comprises at least one defluorinating plasma treatment selected from the group consisting of oxygen plasma treatments, hydrogen-ammonia treatments, ammonia-butadiene-ammonia (ABA) treatments, hydrogen-ammonia-butadiene-ammonia (HABA) treatments and combinations thereof.

24. The method of claim 22 wherein the step of plasma treatment is conducted in the presence of a gas or mixture of gases.

25. The method of claim 24 wherein the gas or mixture of gases is selected from hydrogen, hydrogen in an inert gas, hydrogen in argon and mixtures thereof.

26. The method of claim 22 wherein the plasma treatment utilizes an electric discharge frequency of about 13.56 MHz between about 100-1000 watts at a pressure of about 0.1 -1.0 torr.

27. The method of claim 22 wherein the plasma treatment utilizes an electric discharge frequency of about 13.56 MHz between about 200 to 800 watts.

28. The method of claim 21 further comprising the step of flipping the medical device over to better treat both sides of the lens.

29. The method of claim 22 wherein the plasma-treatment gas is suitably provided at a flow rate of 50 to 500 sccm.

30. The method of claim 22 wherein the plasma-treatment gas is suitably provided at a flow rate of 100 to 300 sccm.

31. The method of claim 21 wherein the medical device is supported within a vessel on a support device designed to adjust the position of the medical devices.

32. The method of claim 21 wherein the medical devices may be provided the surface functionality by placing them, in their unhydrated state, within an electric glow discharge reaction vessel.

33. The method of claim 22 wherein the medical device comprises a hydrogen-plasma treated fluorinated polymeric surface which is subsequently oxidized by an oxidizing plasma.

34. The method of claim 33 wherein the oxidizing plasma is selected from the group consisting of oxygen gas, water, peroxide, air, ammonia, methanol, acetone, alkylamines and mixtures thereof.

35. The method of claim 21 wherein the medical device formed is selected from the group consisting of heart valves, intraocular lenses, contact lenses, intrauterine devices, vessel substitutes, artificial ureters and artificial breast tissue.

36. The method of claim 35 wherein the medical device formed is a contact lens.

37. The method of claim 36 wherein the medical device formed is a soft contact lens.

38. A surface modified medical device comprising:
a medical device manufactured from a monomer mixture conprising surface functionality after polymerization of the monomer mixture; and
one or more reactive terminal functionalized surfactants applied to the surface of the medical device;
whereby a chemical reaction between the surface functionality of the medical device and the one or more reactive terminal functionalized surfactants forms covalent bonds there between.

39. The surface modified medical device of claim 38 wherein the medical device is a contact lens.

40. The surface modified medical device of claim 38 wherein the medical device is a hydrophilic contact lens.

41. The surface modified medical device of claim 38 wherein the medical device is a hydrogel contact lens.

42. The method of claim 1 wherein the step of subjecting the device surface and surface modifying agent to reaction conditions suitable for forming a covalent bond between the device surface and the surface modifying agent to form a surface modified medical device occurs under autoclave conditions.

43. The method of claim 42 further comprising the step of applying a lid stock to a package containing the device and the surface modifying agent prior to subjecting it to the step of autoclaving.

44. The method of claim 43 further comprising the steps of removing the surface modifying agent after autoclaving, rinsing the coated device, providing a storage solution and further autoclaving the device to sterilize the device.

45. The method of claim 21 wherein the step of subjecting the device surface and surface modifying agent to reaction conditions suitable for forming a covalent bond between the device surface and the surface modifying agent to form a surface modified medical device occurs under autoclave conditions.

46. The method of claim 45 further comprising the step of applying a lid stock to a package containing the device and the surface modifying agent prior to subjecting it to the step of autoclaving.

47. The method of claim 45 further comprising the steps of removing the surface modifying agent after autoclaving, rinsing the coated device, providing a storage solution and further autoclaving the device to sterilize the device.

48. A method of inhibiting the attachment of bacteria to a medical device, the method comprising the method of claim 1.

49. A method of inhibiting the attachment of bacteria to a device, the method comprising the method of claim 1.

50. A method of inhibiting the attachment of bacteria to a device, the method comprising the method of claim 21.

51. The surface modified device of claim 38 wherein the device is resistant to the attachment of bacteria.

52. The method of claim 1 wherein the monomer mixture comprises vinal acid, N-vinyl pyrollidinone, vinyl carbonate terminated PDMS, 3-[tris(trimethylsiloxy)silyl]propylvinyl carbamate.

53. The method of claim 1 wherein the monomer mixture comprises diluent, vinal acid, N-vinyl pyrollidinone, fluorine containing vinyl carbonate terminated PDMS, 3-[tris(trimethylsiloxy)silyl]propylvinyl carbamate.

54. The method of claim 1 wherein the monomer mixture comprises methacrylic acid, neopentyl glycol dimethacrylate, methyl methacrylate, N-vinyl pyrollidinone, methacrylate terminated PDMS, 3-[tris(trimethylsiloxy)silyl]propyl methacrylate, bis-hexafluoroitaconate and hexafluoroisopropyl methacrylate.
